(19) United States
(12) Patent Application Publication
(10) Pub. No.: US 2013/0324815 A1
(43) Pub. Date: Dec. 5, 2013

(54) CONTINUOUS MEASUREMENT OF TOTAL HEMOGLOBIN

(71) Applicants: Zhongping Jian, Irvine, CA (US); Clayton M. Young, Redmond, WA (US); Feras Hatib, Irvine, CA (US)

(72) Inventors: Zhongping Jian, Irvine, CA (US); Clayton M. Young, Redmond, WA (US); Feras Hatib, Irvine, CA (US)

(73) Assignee: Edwards Lifesciences Corporation, Irvine, CA (US)

(21) Appl. No.: 13/878,418

(22) PCT Filed: Oct. 4, 2011
(86) PCT No.: PCT/US11/54714

§ 371 Date: Jul. 16, 2013

Related U.S. Application Data
(60) Provisional application No. 61/391,414, filed on Oct. 8, 2010.

(51) Int. Cl. A61B 5/1459

(52) U.S. Cl. CPC A61B 5/1459
USPC 600/327

(57) ABSTRACT

The present application relates to continuous measurement of total hemoglobin (tHb) in whole blood. In one embodiment, different wavelengths are used for normalization of the spectral intensity and calculation of the total hemoglobin. In particular, for normalization, a first wavelength is used wherein the wavelength is substantially insensitive to changes in levels of hemoglobin and oxygen saturation. For calculation of the total hemoglobin, a second wavelength is used. The second wavelength is sensitive to changes in levels of hemoglobin, but substantially insensitive to changes in levels of oxygen saturation. In another embodiment, a continuous measurement can be made using two wavelengths that are both sensitive to oxygen saturation, but they both are equally sensitive. In other words, the normalized intensities associated with the two wavelengths change equal amounts with equal changes in oxygen saturation levels.

### FIELD

The present application relates to measurements of properties of blood and, particularly, to the measurement of total hemoglobin.

### BACKGROUND

Accurate measurement of total hemoglobin (tHB) in whole blood is desirable, especially in critical care units and operating rooms. When tHb concentrations are within normal ranges, the blood effectively delivers adequate oxygen from the lungs to the body's tissues and returns carbon dioxide from the tissues to the lungs. Patients having abnormal levels of tHb can suffer from anemia, loss of blood, nutritional deficiency, and bone marrow disorders. Accurate and efficient measurement of tHb can be a helpful diagnostic procedure in detecting and managing such maladies and is vitally important in managing critically ill patients.

The tHb is commonly measured, either directly or indirectly, using a variety of diagnostic systems and methods. Typically, expensive hospital or laboratory equipment is used. Blood is first drawn from a patient, the red blood cells are lysed, and the hemoglobin is isolated in solution. The free hemoglobin is then exposed to a chemical containing cyanide, which binds tightly with the hemoglobin molecule to form cyanmethemoglobin. After bonding, light is transmitted through the solution, and the total amount of light absorbed by the solution is measured at a plurality of wavelengths Based upon the total amount of light absorbed by the solution, the tHb is determined using the Lambert-Beer law. While well established, the tHb measurement procedure is slow and expensive. And the procedure needs to be repeated anew for each subsequent tHb measurement.

Continuous tHb measurements have been disclosed in WO 2007/033318, published in March 2007. This publication represents an improvement over prior methods. While effective, there is always room for improvement. In particular, the method used in the continuous tHb measurement requires a correction for oxygen saturation. Such a correction has led to some overall inaccuracies.

Various other non-invasive and invasive tHb measurement procedures have been employed. Few, if any, provide maximum accuracy, efficiency, and convenience to patients and healthcare professionals. Therefore, a need exists for systems and methods that increase the accuracy, efficiency, and convenience of tHb measurements for patients.

### SUMMARY

The present application relates to continuous total hemoglobin (tHb) measurement.

In one embodiment, light is projected into blood in a patient and a resultant spectral intensity is obtained. Different wavelengths are used for normalization of the spectral intensity and calculation of the total hemoglobin. In particular, for normalization, a first wavelength is used wherein the wavelength is substantially insensitive to changes in levels of hemoglobin and oxygen saturation. For calculation of the total hemoglobin, a second wavelength is used. The second wavelength is sensitive to changes in levels of hemoglobin, but substantially insensitive to changes in levels of oxygen saturation. Example wavelengths include 800 nm for the first wavelength and 505 nm for the second wavelength, but other wavelengths can be used. This method can be repeated at any desired wavelength to continuously measure total tHb.

In another embodiment, an elevation can be subtracted from the spectral intensity in order to compensate for blood-vessel wall artifacts. To calculate an amount to subtract, a region of wavelengths in the spectral intensity can be selected based on a determination that the region is affected by blood vessel wall artifacts. A minimum intensity in this region can be determined and subtracted from the spectral intensity for each wavelength in the spectrum, other than the predetermined first wavelength. A typical region includes the spectrum between the wavelengths of 400 nm and 600 nm. In this region, a minimum spectral intensity is determined and such a value is used to remove elevation across the spectrum where the blood vessel wall artifacts are present.

In another embodiment, continuously determining the total hemoglobin includes continuously determining hematocrit, as there is a simple linear relationship between the two. For example, under normal conditions, hemoglobin is around 33% of hematocrit. Other estimations can be used.

In another embodiment, a continuous measurement can be made using two wavelengths that are both sensitive to oxygen saturation, but they both are equally sensitive. In other words, the normalized intensities associated with the two wavelengths change equal amounts with equal changes in oxygen saturation levels.

The foregoing and other objects, features, and advantages of the invention will become more apparent from the following detailed description, which proceeds with reference to the accompanying figures.

### BRIEF DESCRIPTION OF THE DRAWINGS

FIG. 1 is an example apparatus that can be used to continuously measure total hemoglobin.

FIG. 2 is an example controller used in FIG. 1.

FIG. 3 is a flowchart of a method for measuring total hemoglobin according to one embodiment.

FIG. 4 is a flowchart of a method for measuring total hemoglobin according to another embodiment.

FIG. 5 is an example showing filtering spectral data.

FIG. 6 is an example showing removing elevation to minimize artifacts in the spectral data.

FIG. 7 is an example plot of normalized intensity data versus wavelength.

FIG. 8 is an example plot of normalized intensity versus wavelength for multiple hemoglobin levels.

FIG. 9 is an example plot used to obtain predetermined coefficients.

FIG. 10 is a flowchart of a method to determine coefficients used to calculate total hemoglobin.

FIG. 11 is a flowchart of an alternative method used to determine total hemoglobin.

FIGS. 12 and 13 show alternative embodiments used for a light source.

### DETAILED DESCRIPTION

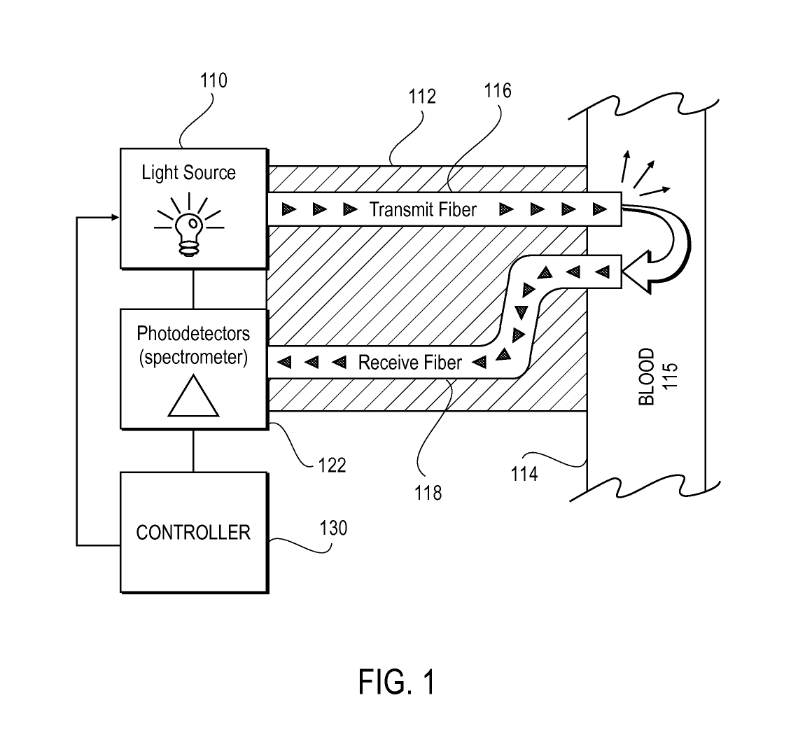

FIG. 1 shows an apparatus used to continuously calculate total hemoglobin. A light source 110 is coupled to a catheter 112 inserted into a blood vessel 114. The light source 110 can be any of a variety of types, such as an LED, and typically produces light in a wavelength range between about 400 nm to about 800 nm. Other light sources can be used. Generally, the light source is turned on continuously over a discrete period of time and generates a plurality of wavelengths that are transmitted into blood 115. The catheter 112 can also be any of a variety of types, such as a central venous catheter or a pulmonary artery catheter, and can include two parallel optical fibers 116, 118. The first optical fiber 116 is a transmit fiber designed to receive light from the light source and project the light into the blood stream illuminating the blood. The second optical fiber 118 is a receive fiber capable of receiving light from the blood and delivering the light to photodetectors 122, which can be included in a spectrometer or other instrument for measuring the properties of light. Although any photodetectors can be used, the photodetectors 122 should preferably be capable of measuring intensities within the range of between about 400 nm and 1000 nm or higher. The received light is generally a combination of reflected light, scattered light and/or light transmitted through the blood. In any event, the received light carries information used to obtain parameters needed for hemodynamic monitoring, such as total hemoglobin and oxygen saturation. Ideally, the light interacts only with the blood. But, in practice, the light interacts not only with the blood, but with other objects located in the environment in which the catheter is positioned, such as blood-vessel wall artifacts.

A controller 130 can be coupled to the photodetectors 122 and associated instrumentation for measuring light intensity. The controller can also be coupled to the light source 110 in order to control the light source during measurements. As further described below, the controller can use the measured light intensity captured in the photodetectors 122 to determine a level of hemoglobin in the blood. Various techniques for using light intensity to determine hemoglobin levels are described further below.

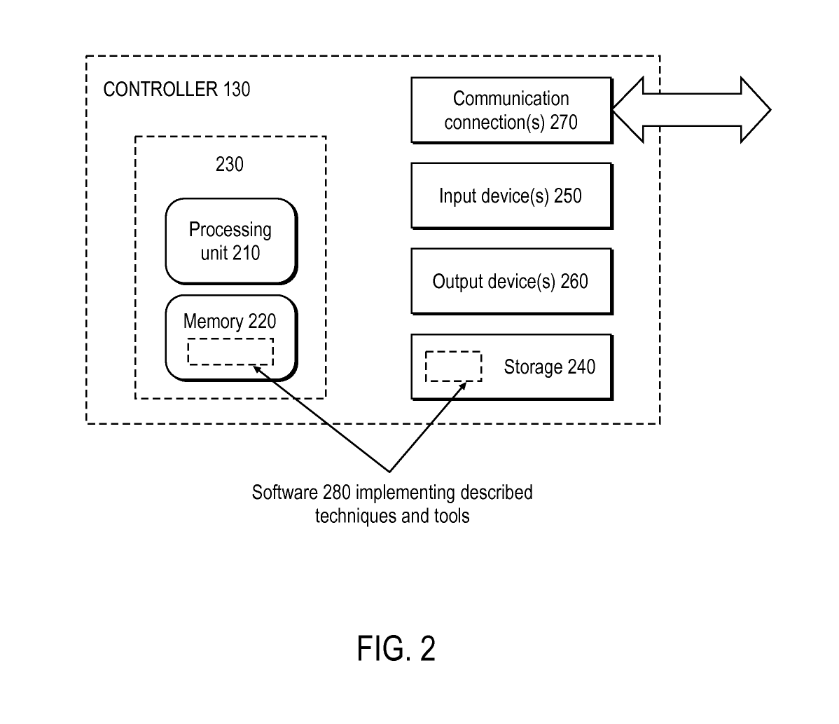

FIG. 2 illustrates a generalized example of a suitable controller 130 in which the described technologies can be implemented. The controller is not intended to suggest any limitation as to scope of use or functionality, as the technologies may be implemented in diverse general-purpose or special-purpose computing environments.

With reference to FIG. 2, the controller 130 can include at least one processing unit 210 (e.g., signal processor, microprocessor, ASIC, or other control and processing logic circuitry) coupled to memory 220. The processing unit 210 executes computer-executable instructions and may be a real or a virtual processor. The memory 220 may be volatile memory (e.g., registers, cache, RAM), non-volatile memory (e.g., ROM, EEPROM, flash memory, etc.), or some combination of the two. The memory 220 can store software 280 implementing any of the technologies described herein.

The controller may have additional features. For example, the controller can include storage 240, one or more input devices 250, one or more output devices 260, and one or more communication connections 270. An interconnection mechanism (not shown), such as a bus or network interconnects the components. Typically, operating system software (not shown) provides an operating environment for other software executing in the controller and coordinates activities of the components of the controller.

The storage 240 may be removable or non-removable, and can include magnetic disks, magnetic tapes or cassettes, CD-ROMs, CD-RWs, DVDs, or any other computer-readable media that can be used to store information and which can be accessed within the controller. The storage 240 can store software 280 containing instructions for detecting blood-vessel wall artifacts associated with a catheter position in a blood-vessel wall.

The input device(s) 250 can be a touch input device such as a keyboard, mouse, pen, or trackball, a voice input device, a scanning device, or another device. The output device(s) 260 may be a display, printer, speaker, CD- or DVD-writer, or another device that provides output from the controller. Some input/output devices, such as a touchscreen, may include both input and output functionality.

The communication connection(s) 270 enables communication over a communication mechanism to another computing entity. The communication mechanism conveys information such as computer-executable instructions, audio/video or other information, or other data. By way of example, and not limitation, communication mechanisms include wired or wireless techniques implemented with an electrical, optical, RF, microwave, infrared, acoustic, or other carrier.

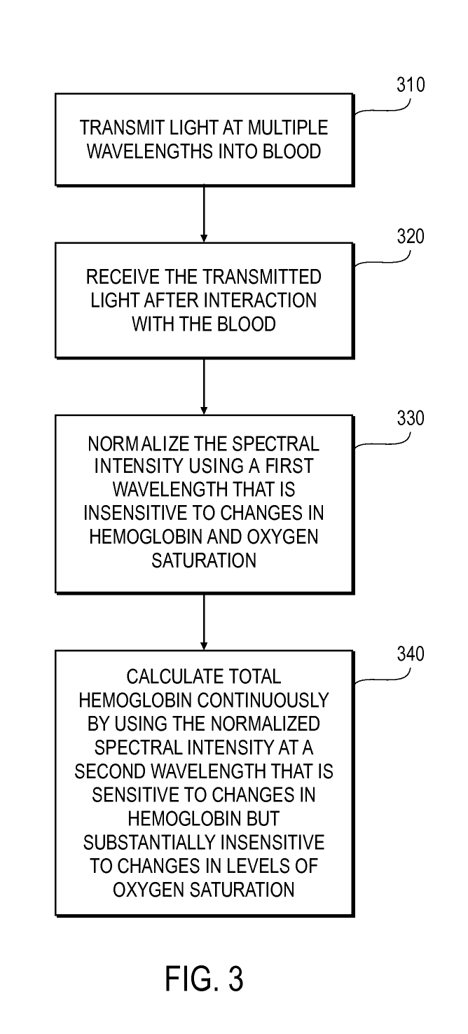

FIG. 3 is a flowchart of a method for continuous measurement of total hemoglobin. In process block 310, light is transmitted into the blood to be measured at multiple wavelengths. For example, in the embodiment shown in FIG. 1, the transmit fiber 116 can be used to transmit light from a light source 110. In process block 320, light is received after interaction with the blood. Light waves that interact with blood can include reflected light, scattered light, and/or transmitted light. The receive fiber 118 and photodetectors 122 are examples of a structure that can be used to receive the light. In any event, a spectral intensity is obtained based on the received light after interaction with the blood. In process block 330, the spectral intensity is normalized. Normalization refers to using a reference wavelength to divide the spectral data to bring all data to a common scale. The reference wavelength used should be substantially insensitive to changes in levels of hemoglobin and oxygen saturation. By substantially insensitive, it is meant that there can be insignificant changes in intensity levels with changes in levels of hemoglobin and oxygen saturation, but such insignificant changes have little impact on the overall measurement of hemoglobin. In process block 340, the total hemoglobin is calculated continuously using the normalized intensity at a predetermined wavelength. The predetermined wavelength is a different wavelength from that used in the normalization. In particular, the wavelength chosen should be sensitive to changes in levels of hemoglobin, but substantially insensitive to changes in levels of oxygen saturation. An example wavelength for the normalization is 800 nm and an example wavelength for the calculation of total hemoglobin is 505 nm. For the calculation of total hemoglobin, it is desirable that a formula be used with predetermined coefficients. An example formula can be a polynomial. In one very specific example, the following second-order polynomial can be used: tHb=a·(ratio_1)²+b·(ratio_2)+c, wherein a, b, and c are the predetermined coefficients. The ratio_1 and ratio_2 can be equal (derived from the normalized intensity at the same wavelength) or can be different numbers derived from the normalized intensity at different wavelengths. In one embodiment, the ratio_1 and ratio_2 are determined using a base ten logarithm of the normalized intensity at a predetermined wavelength, such as 505 nm. Other wavelengths can be used, but it is desirable to use a wavelength that is sensitive to hemoglobin, but substantially insensitive to changes in levels of oxygen saturation.

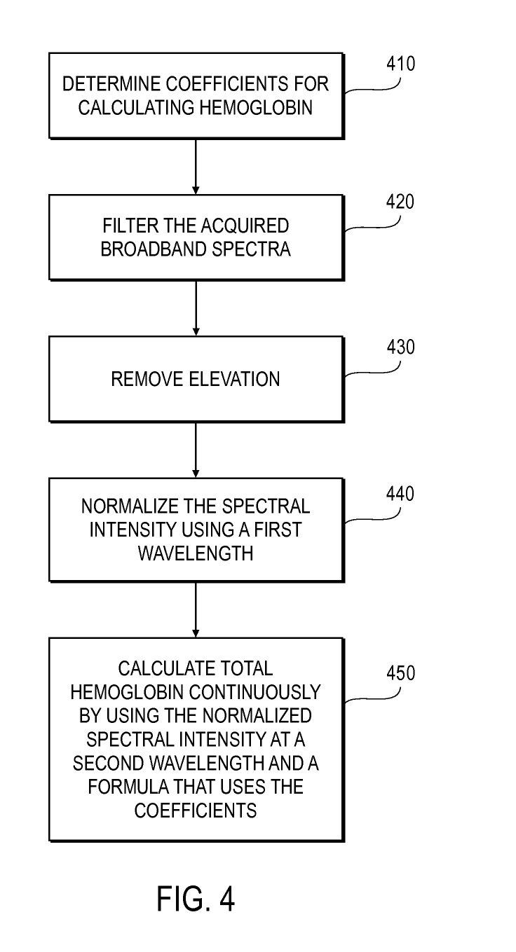

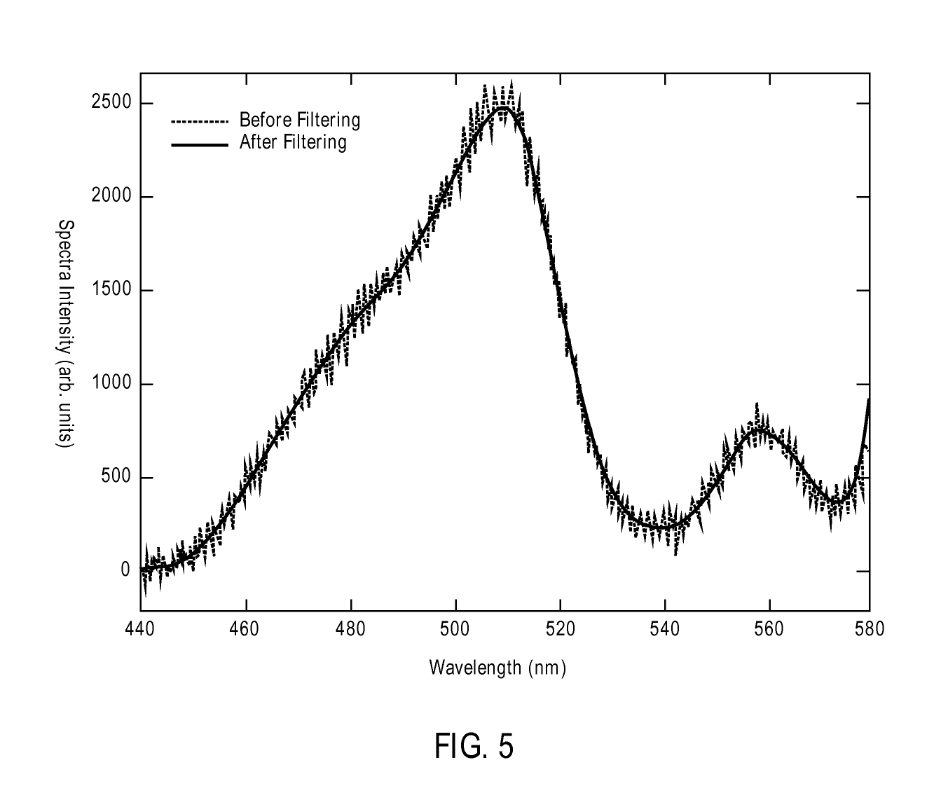

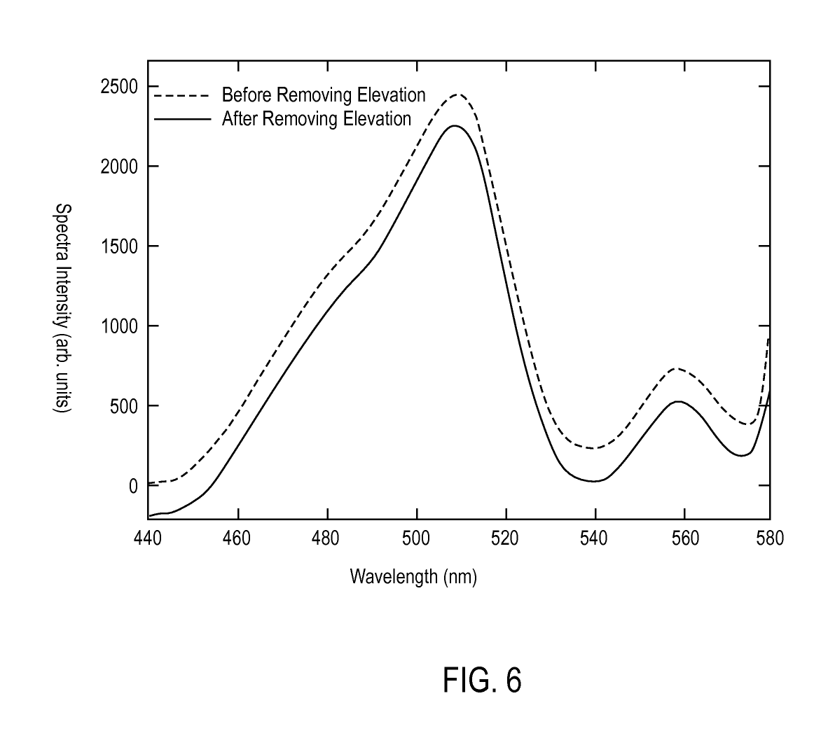

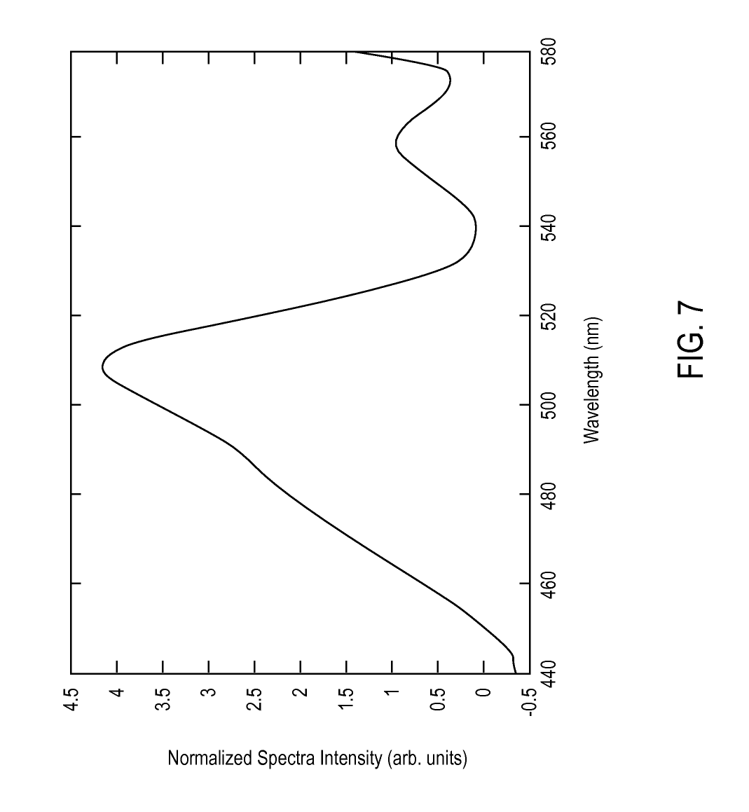

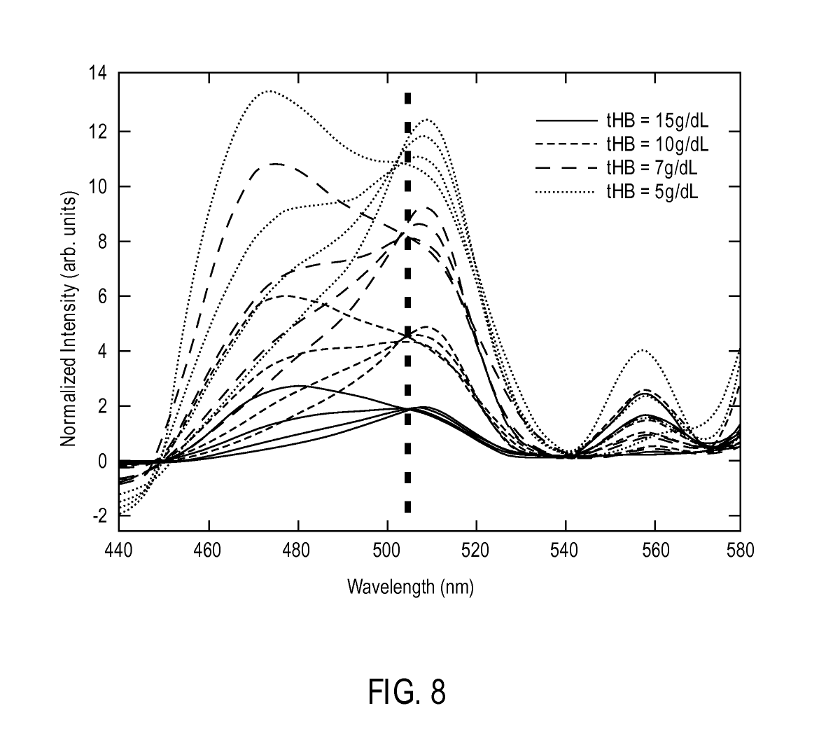

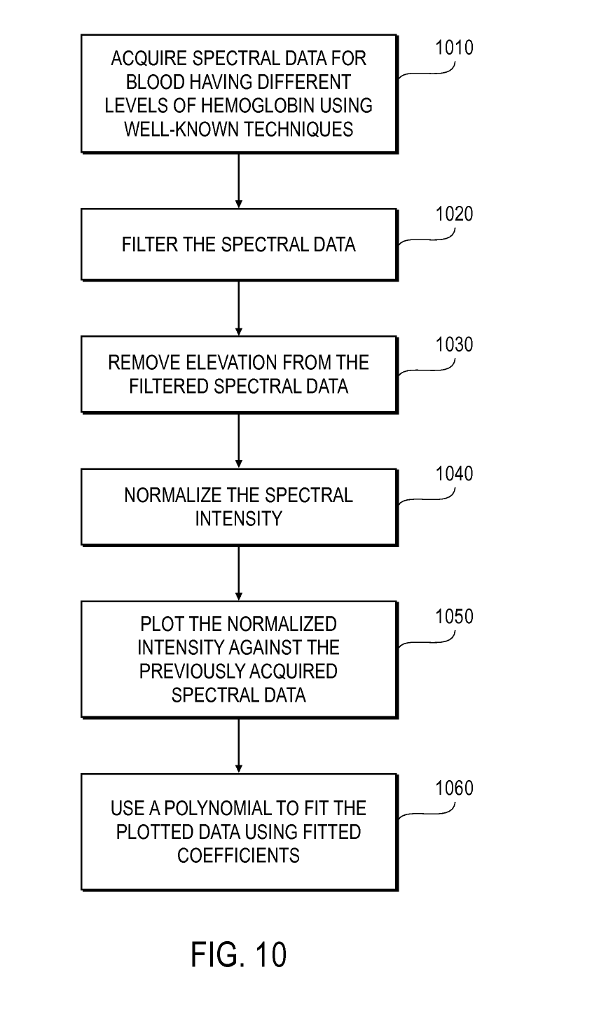

FIG. 4 shows a more detailed flowchart that can be used in one embodiment. In process block 410, predetermined coefficients are calculated. The predetermined coefficients can be calculated by obtaining spectral data for multiple blood samples having different levels of hemoglobin and processing the spectral data using process blocks 420, 430, 440 and 450, as outlined below. FIG. 10 also discusses a specific embodiment for calculation of the coefficients. In process block 420, broadband spectra that are acquired through the catheter of FIG. 1 are filtered to attenuate noise (e.g., background and random noise.) FIG. 5 shows a specific example of data before and after filtering. In process block 430, the elevation is removed. Removing elevation is beneficial to compensate for artifacts introduced by a blood-vessel wall. To remove elevation, a region of wavelengths is selected that are affected by the blood-vessel wall artifacts. A minimum intensity value is determined in the selected region, and the minimum intensity value is subtracted from the spectral intensity on a per-wavelength basis. Other techniques for attenuating artifacts of a blood-vessel wall can also be used. FIG. 6 shows a plot of spectral intensity versus wavelength and shows before and after views with elevation removed. In process block 440, the spectral intensity is normalized using a first wavelength. FIG. 7 shows an example of normalization with all wavelengths of the spectral intensity (with elevation removed) divided by the spectral intensity at the wavelength of 800 nm. In process block 450, the total hemoglobin can be calculated using a second wavelength. An example second wavelength that can be used is one that is isosbestic and sensitive to changes in levels of hemoglobin. For example, FIG. 8 shows that the wavelength 505 nm is isosbestic. Specifically, for the same levels of hemoglobin and varying levels of oxygen saturation, the plots converge at the wavelength of 505 nm. Using such a wavelength provides accurate results.

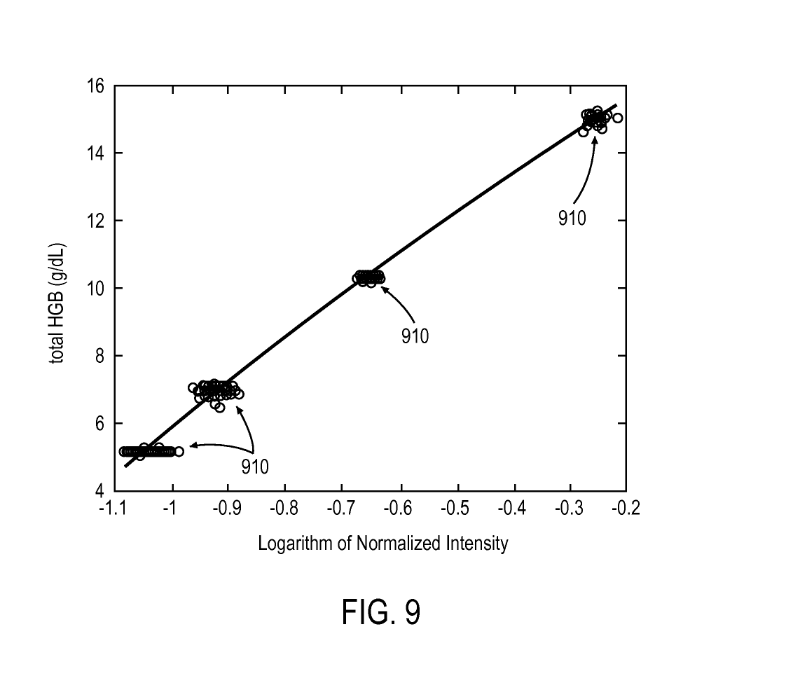

FIG. 10 is a flowchart of a method for calculating coefficients, which, in turn, can be used to calculate total hemoglobin (e.g., process block 340 of FIG. 3.) In process block 1010, the spectral data is acquired for blood having different levels of hemoglobin using well-known techniques. For example, a gold standard method of Instrument Laboratory® can be used. The acquired spectral data is then processed using the techniques already described. For example, the spectral data can be filtered (process block 1020) and the elevation removed therefrom (process block 1030). In process block 1040, the spectral intensity is then normalized using any of the techniques already described. In process block 1050, a plot is generated using a base 10 logarithm of the normalized intensity data against the previously acquired data (see FIG. 9 at 910.) At process block 1060, a polynomial function is generated that best fits (e.g., least squares fit) the data, and the coefficients are generated therefrom. FIG. 9 shows the resultant plot.

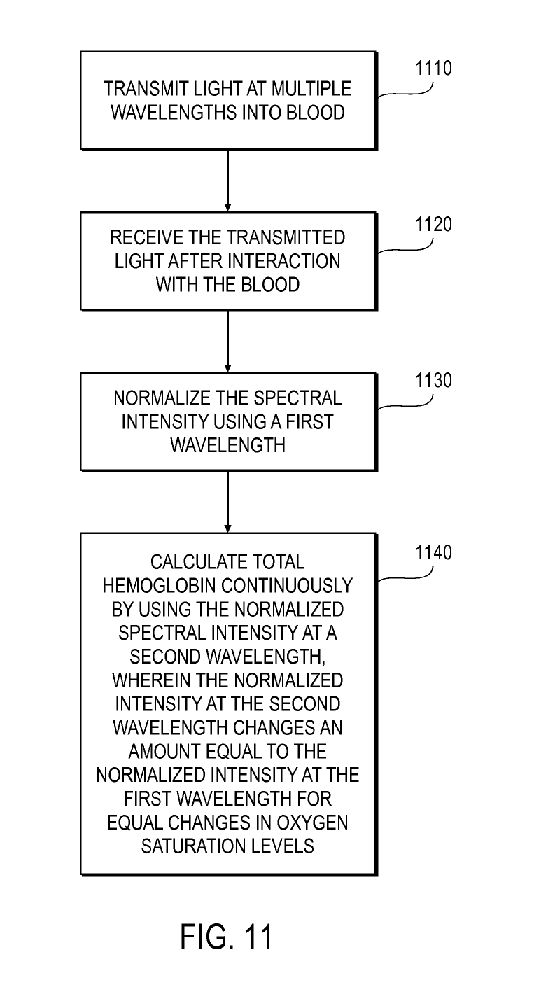

FIG. 11 shows another embodiment that can be used. In process blocks 1110 and 1120, light is transmitted into blood and received using a catheter as already described. In process block 1130, spectral data is acquired from the received light and normalized using a first wavelength, as already described. In process block 1140, the total hemoglobin can be calculated using the normalized spectral intensity at a second wavelength, wherein the normalized intensity at the second wavelength changes an amount equal to the normalized intensity at the first wavelength for equal changes in oxygen saturation levels.

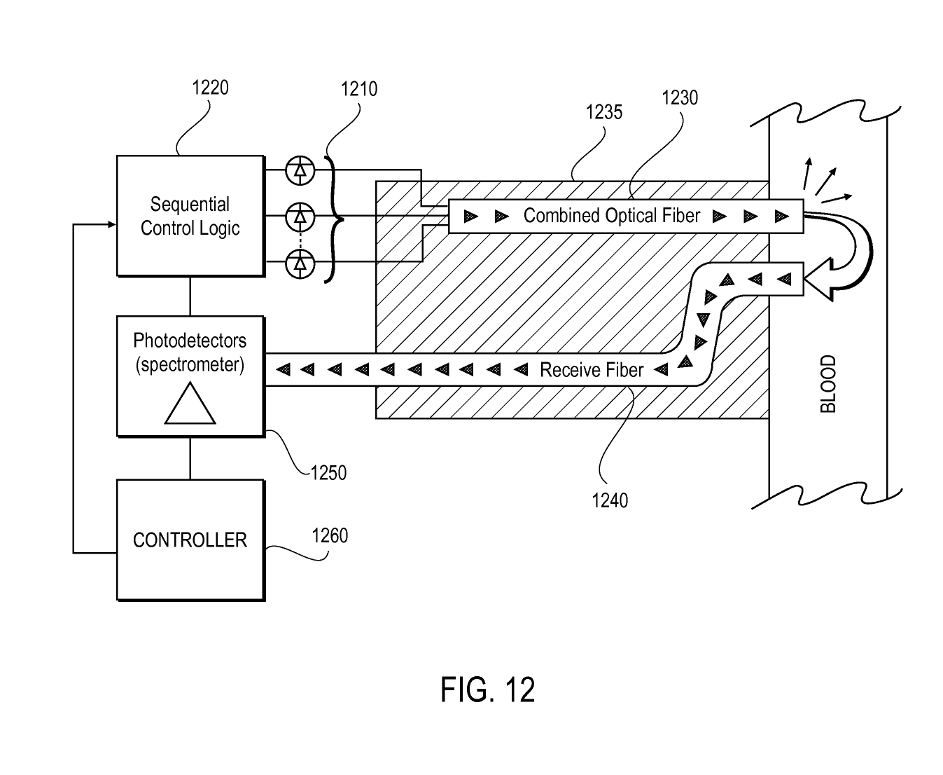

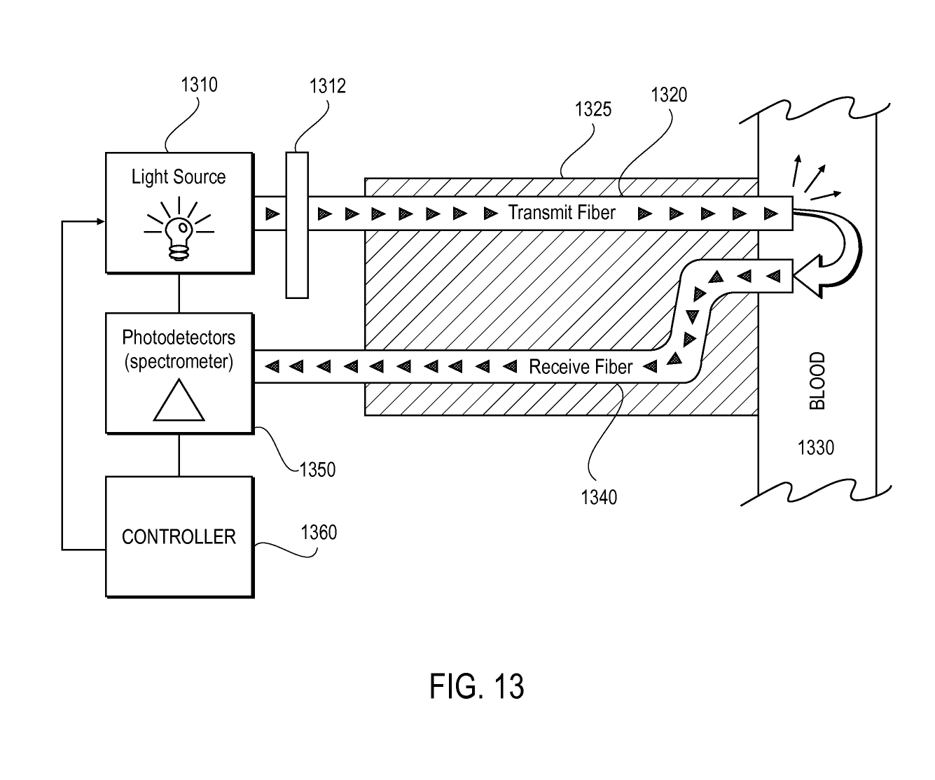

FIGS. 12 and 13 show other structures that can be used to implement the methods described herein. In FIG. 12, multiple light sources 1210, such as multiple colored LEDs can be used to provide discrete wavelengths that can be time multiplexed by sequencer control logic 1220 to individually turn on at different times. The discrete signals are transmitted through an optical transmit fiber 1230 located in a catheter 1235 into the blood and reflected into a receive fiber 1240. The receive fiber 1240 transmits the discrete reflected signals to a single photodetector of a spectrometer 1250. Multiple photodetectors may be employed to measure the special effects of the signals. A controller 1260 is coupled to the photodetectors and is used to determine blood-vessel wall artifacts and/or catheter tip location, as previously described.

In FIG. 13, single or multiple light sources 1310 may be transmitted through a wavelength filter 1312, such as a filter wheel, to provide an alternate or additional embodiment of discrete wavelengths that may be time multiplexed. The light signals are passed through the filter 1312 and transmitted through an optical fiber 1320 located in a catheter 1325 into blood 1330 and then reflected back through a receive fiber 1340 to at least one photodetector 1350. A controller 1360 is coupled to the photodetectors and is used to determine blood-vessel wall artifacts and/or catheter tip location, as previously described.

The techniques herein can be described in the general context of computer-executable instructions, such as those included in program modules, being executed in a computing environment on a target real or virtual processor. Generally, program modules include routines, programs, libraries, objects, classes, components, data structures, etc., that perform particular tasks or implement particular abstract data types. The functionality of the program modules may be combined or split between program modules as desired in various embodiments. Computer-executable instructions for program modules may be executed within a local or distributed computing environment.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

Any of the disclosed methods can be implemented as computer-executable instructions stored on one or more computer-readable storage media (e.g., non-transitory computer-readable media, such as one or more optical media discs, volatile memory components (such as DRAM or SRAM), or nonvolatile memory components (such as hard drives)) and executed on a computer (e.g., any commercially available computer, including smart phones or other mobile devices that include computing hardware). Any of the computer-executable instructions for implementing the disclosed techniques as well as any data created and used during implementation of the disclosed embodiments can be stored on one or more computer-readable media (e.g., non-transitory computer-readable media). The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Pert, JavaScript, Adobe Flash, or any other suitable programming language. Likewise, the disclosed technology is not limited to any particular computer or type of hardware. Certain details of suitable computers and hardware are well known and need not be set forth in detail in this disclosure.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope of these claims.

We claim:

1. A method of determining total hemoglobin of blood, comprising:
transmitting light of multiple wavelengths into the blood using a catheter;
receiving the light after interacting with the blood;
normalizing a spectral intensity of wavelengths of the received transmitted light using a first predetermined wavelength, wherein an intensity at the first predetermined wavelength is substantially insensitive to changes in levels of hemoglobin and oxygen saturation;
calculating the total hemoglobin of the blood using the normalized spectral intensity at a second predetermined wavelength, wherein the normalized intensity at the second predetermined wavelength is sensitive to changes in levels of hemoglobin, but substantially insensitive to changes in levels of oxygen saturation.

2. The method of claim 1, wherein the catheter includes a transmit optical fiber and a receive optical fiber.

3. The method of claim 1, wherein measuring the intensity includes receiving at least one light wavelength from a receive optical fiber and using a photodetector to capture electromagnetic energy associated therewith.

4. The method of claim 1, further including filtering the spectral intensity to attenuate noise.

5. The method of claim 1, further including removing elevation of intensity to compensate for blood-vessel wall artifacts.

6. The method of claim 5, wherein removing the elevation includes selecting a region of wavelengths affected by blood vessel wall artifacts, determining a minimal intensity value in the selected region and subtracting the minimal intensity value from the spectral intensity.

7. The method of claim 6, wherein the region of wavelengths is between 400 nm and 600 nm.

8. The method of claim 1, wherein calculating the total hemoglobin includes using a polynomial with predetermined coefficients.

9. The method of claim 8, wherein the polynomial includes the formula tHb=a·(ratio_1)²+b·(ratio_2)+c, wherein a, b, and c are the predetermined coefficients.

10. The method of claim 9, wherein ratio_1 and ratio_2 are calculated using the same wavelength.

11. The method of claim 9, wherein ratio_1 and ratio_2 are calculated using different wavelengths

12. The method of claim 9, wherein the coefficients are calculated by obtaining spectra data for multiple blood samples having different levels of hemoglobin and using the method of claim 1 to process the spectra data

13. The method of claim 12, wherein the resulting processed spectra data is plotted using a logarithmic scale and a linear least squares fitting technique.

14. The method of claim 1, wherein the first predetermined wavelength is 800 nm and the second predetermined wavelength is 505 nm.

15. A computer-readable storage medium having instructions encoded thereon operable to cause a computer to perform the method of claim 1.

16. An apparatus for determining total hemoglobin of blood, comprising:
a catheter including a transmit optical fiber and a receive optical fiber;
a light source coupled to the transmit optical fiber for transmitting light into blood;
one or more photodetectors coupled to the receive optical fiber for receiving the light after it interacts with the blood; and
a controller coupled to the one or more photodetectors for receiving a spectral intensity of one or more wavelengths and for normalizing the spectral intensity of the wavelengths using a first predetermined wavelength, wherein an intensity at the first predetermined wavelength is substantially insensitive to changes in levels of hemoglobin and oxygen saturation and for calculating the total hemoglobin of the blood using the normalized spectral intensity at a second predetermined wavelength, wherein the normalized intensity at the second predetermined wavelength is sensitive to changes in levels of hemoglobin, but substantially insensitive to changes in levels of oxygen saturation.

17. A method of determining total hemoglobin of blood, comprising:
transmitting light at multiple wavelengths into the blood using a catheter;
receiving the light after interacting with the blood;
normalizing a spectral intensity of wavelengths of the received transmitted light using a first predetermined wavelength;
calculating the total hemoglobin of the blood using the normalized spectral intensity at a second predetermined wavelength, different from the first predetermined wavelength, wherein the normalized intensity at the second predetermined wavelength changes an amount equal to the normalized intensity of the first predetermined wavelength with equal changes in the oxygen saturation levels.
